(19) United States
(12) Patent Application Publication
(10) Pub. No.: US 2009/0312562 A1
(43) Pub. Date: Dec. 17, 2009

(54) Catalyst Containing Titanium Dioxide, Particularly for the Production of Phthalic Anhydride

(75) Inventors: Christian Guckel, Grafing (DE); Marvin Estenfelder, Karlsruhe (DE); Gerhard Mestl, Munchen (DE)

Correspondence Address: SCOTT R. COX;LYNCH, COX, GILMAN & MAHAN, P.S.C., 500 WEST JEFFERSON STREET, SUITE 2100, LOUISVILLE, KY, 40202, US

(73) Assignee: SUD-CHEMIE AG, Munchen (DE)

(21) Appl. No.: 12/301,325

(22) PCT Filed: May 23, 2007
(86) PCT No.: PCT/EP07/04569

§ 371 Date: Nov. 18, 2008

(30) Foreign Application Priority Data
May 23, 2006 (EP) 0601068.2

(51) Int. Cl. C07D 307/89; B01J 21/06; B01J 31/38; B01J 8/04; C01G 23/047

(52) U.S. Cl. 549/249; 502/350; 502/159; 422/191; 423/610

(57) ABSTRACT

The present invention relates to the use of titanium dioxide having a content of sulphur, calculated as elemental sulphur, of less than about 1000 ppm and a BET surface area of at least 5 m²/g for preparing a catalyst for gas phase oxidation of hydrocarbons, especially for gas phase oxidation of o-xylene and/or naphthalene. Also described is a preferred process for preparing such a catalyst.

The invention relates to a catalyst comprising titanium dioxide, especially for preparing phthalic anhydride (PA) by gas phase oxidation of o-xylene and/or naphthalene. In a preferred aspect, the present invention relates to the use of titanium dioxide with minor impurities of sulphur, and preferably a minimum content of niobium, for preparation of and in catalysts for gas phase oxidation of hydrocarbons.

The industrial scale production of phthalic anhydride is achieved by the catalytic gas phase oxidation of o-xylene and/or naphthalene. For this purpose, a catalyst suitable for the reaction is charged into a reactor, preferably what is known as a tube bundle reactor in which a multitude of tubes are arranged in parallel, and is flowed through from the top or bottom with a mixture of the hydrocarbon(s) and an oxygenous gas, for example air. Owing to the intense heat formation of such oxidation reactions, it is necessary for a heat carrier medium to flow around the reaction tubes to prevent what are known as hotspots and thus to remove the amount of heat formed. This energy may be utilized for the production of steam. The heat carrier medium used is generally a salt melt and here preferably a eutectic mixture of NaNO₂ and KNO₃.

To suppress the unwanted hotspots, it is likewise possible to charge a structured catalyst into the reaction tube, as a result of which, for example, two or three catalyst zones composed of catalysts of different composition can arise. Such systems are as such already known from EP 1 082 317 B1 or EP 1 084 115 B1.

The layer-by-layer arrangement of the catalysts also has the purpose of keeping the content of undesired by-products, i.e. compounds which are before the actual product of value in a possible reaction mechanism of o-xylene and/or naphthalene to phthalic anhydride, in the crude PA as low as possible. These undesired by-products include mainly the compounds o-tolylaldehyde and phthalide. The further oxidation of these compounds to phthalic anhydride additionally increases the selectivity for the actual product of value.

In addition to the above-addressed under-oxidation products, over-oxidation products also occur in the reaction. These include maleic anhydride, citraconic anhydride, benzoic acid and the carbon oxides. A selective suppression of the formation of these undesired by-products in favour of the product of value leads to a further increase in the productivity and economic viability of the catalyst.

Corresponding considerations also apply in the case of other catalysts, for example for the partial oxidation of other hydrocarbons.

There is a constant need for catalysts which have a high conversion at high selectivity, and thus enable an improved productivity and economic viability.

It was therefore an object of the present invention to develop a catalyst or a catalyst system which avoids the disadvantages of known catalysts from the prior art and enables an improvement in the activity, selectivity and/or lifetime of the catalyst.

Accordingly, a first aspect of the invention relates to the use of titanium dioxide having a content of sulphur, calculated as elemental sulphur, of less than about 1000 ppm for preparing a catalyst for gas phase oxidation of hydrocarbons. The catalyst comprises the titanium dioxide preferably in the catalytically active composition.

It the context of the present invention, it has been found that, surprisingly, the use of TiO₂ having a content of sulphur, calculated as elemental sulphur, of less than 1000 ppm leads to improved catalysts for the gas phase oxidation of hydrocarbons, said catalysts enabling, for example in the gas phase oxidation of o-xylene and/or naphthalene to phthalic anhydride, an improved C₈ selectivity and an advantageous low COₓ selectivity of the catalyst with simultaneously improved conversion. An unexpectedly smaller amount of MA (maleic anhydride) was also formed as a by-product in favour of an improved PA selectivity.

This was all the more surprising since the prior art for oxidation catalysts even discloses that a regeneration or activation of the catalysts can be performed via the addition of sulphur trioxide, i.e. the supply of sulphur. For this sector, it was thus familiar to the person skilled in the art that sulphur is not only harmless but, on the contrary, useful for the activity of the catalyst. Vanadium-containing oxidation catalysts are also used conventionally for the preparation of sulphuric acid. Accordingly, WO 03/081481 relates to titanium oxide regeneration processes for Fischer-Tropsch catalysts, i.e. for reactions under reductive conditions at high pressures, in which—in contrast to the present oxidation reactions—the formation of metal sulphides constitutes a problem. The use of a titanium dioxide in catalysts for the gas phase oxidation of hydrocarbons as described and claimed herein cannot be taken from U.S. Pat. No. 5,527,469 either, in which merely a process for preparing desulphurized titanium dioxide hydrolysate with high purity is disclosed.

More preferably, the content of sulphur in the TiO₂ used (calculated as elemental sulphur) is less than about 900 ppm, in particular less than 750 ppm, preferably less than 500 ppm, more preferably less than about 300 ppm.

In the context of the present invention, it has also been found that the advantages of the inventive catalyst comprising TiO₂ with a low impurity of sulphur are exhibited particularly clearly when the TiO₂ has a BET surface area of at least 5 m²/g, in particular of at least 12 m²/g. In the case of the preferred used of the catalyst for the gas phase oxidation of o-xylene and/or naphthalene to phthalic anhydride, the BET surface area (DIN 66131) of the TiO₂ material used is preferably in the range between about 15 and 60 m²/g, in particular between 15 and 45 m²/g, more preferably between 15 and 35 m²/g.

In a further aspect of the present invention, it has also been found that, unexpectedly, a relatively high proportion of niobium in the (low-sulphur) titanium dioxide used offers surprising advantages in catalysts for the gas phase oxidation of hydrocarbons. In a particularly preferred inventive embodiment, the content of niobium (calculated as Nb) of the TiO₂ used is therefore more than about 500 ppm, in particular more than 1000 ppm. It has thus been found that a high activity can be achieved at high selectivity of the catalyst. This is the case, for example, in the gas phase oxidation of o-xylene and/or naphthalene to phthalic anhydride with high catalyst activity and very high C₈ selectivity and phthalic anhydride (PA) selectivity. The preferred context of niobium can be established, for example, through the use of niobic acid or niobium oxalate during the preparation of the TiO₂. It has also been found in the context of the present invention that the low sulphur content and the high niobium content of the titanium dioxide act together advantageously in the properties of the catalyst prepared with it. In the purification process according to WO03/018481 A, owing to the selected treatment conditions, especially the elevated temperature, not only the sulphur but also the niobium is removed from the titanium dioxide. The same applies to the preparation process according to U.S. Pat. No. 5,527,469, which additionally relates to a titanium dioxide precursor, titanium dioxide hydrolysate. Too high a removal of the niobium is, according to the present invention, however, surprisingly disadvantageous.

In a further aspect of the present invention, it has also been found that, unexpectedly, a low content of phosphorus in the TiO₂ used, calculated as elemental phosphorus, enables a particularly advantageous selectivity of the catalyst with very good conversion. Accordingly, in a preferred inventive embodiment, the TiO₂ used has a content of phosphorus, calculated as elemental phosphorus, of less than about 800 ppm, preferably of less than about 700 ppm, in particular less than about 500 ppm, in particular of less than about 300 ppm. In the case of the preparation of phthalic anhydride, an unexpectedly smaller amount of MA (maleic anhydride) was also formed as a by-product in favour of an improved PA selectivity.

More preferably, the TiO₂ used in accordance with the invention has both the low sulphur content and the above-described high niobium content, and, more preferably, also the low phosphorus content as defined above.

In a further aspect of the present invention, it has been found, however, that even TiO₂ materials which have the above low phosphorus content, even in the case of a relatively high sulphur content (more than about 1000 ppm), exhibit a better activity and selectivity than TiO₂ materials which do not have the above low phosphorus content.

According to the invention, at least some of the TiO₂ used in the catalyst has the above specification with regard to the sulphur content and preferably also the niobium content and/or the phosphorus content. However, the inventive catalyst will preferably predominantly, i.e. to an extent of more than 50%, in particular more than 75%, more preferably more than 90%, in particular essentially or completely, comprise only TiO₂ materials with the above specifications. It is also possible to use blends of different TiO₂ materials.

Suitable TiO₂ materials are commercially available or can be obtained by standard processes by the person skilled in the art, provided that it is ensured in the synthesis that the starting reagents and raw materials used contain correspondingly low impurities of sulphur (and preferably also phosphorus), and optionally also already have a niobium content in the desired magnitude. Alternatively, it is also possible to proceed from TiO₂ materials having a relatively high sulphur or phosphorus content, and to establish the range required in accordance with the invention by a suitable washing. For example, it is possible to wash in successive wash steps with 0.1-1 molar nitric acid, bidistilled water, 1 molar aqueous ammonia and then again with bidistilled water. This wash cycle can, if required, also be repeated once or more than once. The duration of the individual wash steps can also be varied. For example, a wash step can be performed for 3 to 16 hours. After each wash step, the material can be removed from the particular wash solution in a conventional manner, for example by filtration, before the next wash step. In order to reduce or to prevent the removal of niobium, the wash steps are preferably not performed at elevated temperature, but rather, for example, at room temperature (20° C.) or lower. After the last wash step, the material can be dried.

A process for determining the content of the impurities in the TiO₂ specified herein, especially the sulphur, phosphorus and niobium contents of the TiO₂ used, is specified below before the Examples (DIN ISO 9964-3).

In a further preferred embodiment, the active composition (catalytically active composition) of the inventive catalyst comprises titanium dioxide having a specific BET surface area and preferably a specific pore radius distribution, on which subject reference is made to the parallel WO 2005/11615 A1 to the same applicant. According to this, preference is given to the use of titanium dioxide in which at least 25%, in particular at least about 40%, more preferably at least about 50%, most preferably at least about 60%, of the total pore volume is formed by pores having a radius between 60 and 400 nm. Moreover, according to this, in a preferred embodiment, TiO₂ which has a primary crystal size (primary particle size) of more than about 210 ångström, preferably more than 250 ångström, more preferably at least 300 ångström, in particular at least about 350 ångström, more preferably at least 390 ångström, is used. It has thus been found that such TiO₂ primary crystals with the aforementioned (minimum) size enable the preparation of particularly advantageous catalysts. The primary crystal size is preferably below 900 ångström, in particular below 600 ångström, more preferably below 500 ångström. The above primary crystal size apparently enables, without the invention being restricted to this assumption, the formation of a not excessively compact but rather open-pored structure of the titanium dioxide in the catalyst. One process for determining the primary crystal size is specified in the method part below.

In a further preferred embodiment, TiO₂ which has a bulk density of less than 1.0 g/ml, in particular less than 0.8 g/ml, more preferably less than about 0.6 g/ml, is used. Most preferred are TiO₂ materials having a bulk density of not more than about 0.55 g/ml. A process for determining the bulk density is specified in the method part below. It has thus been found that the use of titanium dioxide having a bulk density as defined above enables the preparation of particularly high-performance catalysts. It is assumed, without the invention being restricted to this, that the bulk density here is a measure of a particularly favourable structure of the TiO₂ surface area available in the catalyst, and the loose, not excessively compact structure provides particularly favourable reaction spaces and access and exit routes for the reactants and reaction products respectively.

The catalysts prepared with inventive use of the titanium dioxide described herein may be used in various reactions for the gas phase oxidation of hydrocarbons. The expression “gas phase oxidation” also includes partial oxidations of the hydrocarbons. The use for preparing phthalic anhydride by gas phase oxidation of o-xylene, naphthalene or mixtures thereof is especially preferred. However, a multitude of other catalytic gas phase oxidations of aromatic hydrocarbons, such as benzene, xylenes, naphthalene, toluene or durene, is also known for the preparation of carboxylic acids and/or carboxylic anhydrides in the prior art. In these oxidations, for example, benzoic acid, maleic anhydride, isophthalic acid, terephthalic acid or pyromellitic anhydride are obtained. The inventive catalyst can also be used in such reactions.

In the partial oxidation of alcohols to the corresponding aldehydes or/and carboxylic acids, for example the oxidation of methanol to formaldehyde, or carboxylic acids or/and the oxidation of aldehydes to the corresponding carboxylic acids, the use of the inventive catalyst is also advantageous.

Also of interest, for example, is the use in the ammoxidation of alkanes and alkenes, the ammoxidation of alkylaromatics and alkylheteroaromatics to the corresponding cyano compounds, especially the ammoxidation of 3-methylpyridine (β-picoline) to 3-cyanopyridine, in the oxidation of 3-methylpyridine to nicotinic acid, in the oxidation of acenaphthene to naphthalic anhydride, or in the oxidation of durene to pyromellitic anhydride. A preferred use also includes the preparation of naphthalic anhydride from acenaphthene and the preparation of cyanopyridine from alkylpyridine (picoline) by ammoxidation, for example of 3-methylpyridine to 3-cyanopyridine. Examples of the general composition of catalysts and reaction conditions suitable therefor can be found, for example, in Saurambaeva and Sembaev, Eurasian ChemTech Journal 5 (2003), p. 267-270. A review of the (amm)oxidation of methylpyridines can be found, for example, in R. Chuck, Applied Catalysis, A: General (2005), 280(1), 75-82. Further advantageous uses of the inventive catalyst or of the TiO₂ as defined herein relate to oxidation dehydrogenations, for example of ethane, propane, butane, isobutane or longer-chain alkanes to the particular alkenes.

The catalysts, especially for the above-described ammoxidation and oxidation reactions, may, in accordance with the invention, be unsupported catalysts or coated catalysts in the form of the shaped bodies and geometries known to those skilled in the art. It is particularly advantageous when the active composition is applied to an inert support.

In general, in the reaction, a mixture of a gas comprising molecular oxygen, for example air, and the starting material to be oxidized is passed through a fixed bed reactor, especially a tube bundle reactor, which may consist of a multitude of tubes arranged in parallel. In the reactor tubes, a bed of at least one catalyst is disposed in each case. Frequently, a bed of a plurality of (different) catalyst zones is advantageous.

In one aspect, when the catalysts prepared in accordance with the invention are used to prepare phthalic anhydride by gas phase oxidation of o-xylene and/or naphthalene, it was found that, surprisingly, the inventive catalysts afford a high conversion with simultaneously low formation of the undesired by-products COₓ, i.e. CO₂ and CO. Furthermore, very good C₈ and PA selectivities are found, as a result of which the productivity of the catalyst is increased overall. The low COₓ selectivity also gives rise in an advantageous manner to lower heat evolution and lower hotspot temperatures. The result is slower deactivation of the catalyst in the hotspot region.

Unless stated otherwise, the pore volumes and fractions reported herein are determined by means of mercury porosimetry (to DIN 66133). The total pore volume is reported in the present description based in each case on the total pore volume between pore radius size 7500 and 3.7 nm measured by means of mercury porosimetry.

In one possible inventive embodiment, it is also possible for only a portion of the titanium dioxide used for catalyst preparation to have the properties described herein, even though this is generally not preferred. The shape of the catalyst and its homogeneous or heterogeneous structure is also not restricted in principle in the context of the present invention and may include any embodiment which is familiar to those skilled in the art and appears to be suitable for the particular field of use.

In many cases, for instance when the inventive catalyst is used in a particularly preferred embodiment to prepare phthalic anhydride, so-called coated catalysts have been found to be useful. In this context, use is made of a support which is inert under the reaction conditions, for example composed of quartz (SiO₂), porcelain, magnesium oxide, tin dioxide, silicon carbide, rutile, alumina (Al₂O₃), aluminium silicate, magnesium silicate (steatite), zirconium silicate or cerium silicate, or composed of mixtures of the above materials. The support may, for example, have the form of rings, spheres, shells or hollow cylinders. The catalytically active composition is applied thereto in comparatively thin layers (coatings). It is also possible for two or more layers of the identical or different catalytically active composition to be applied.

Depending on the intended use of the inventive catalyst, in addition to the TiO₂ used in accordance with the invention, it is possible for the components customary and familiar to those skilled in the art to be present in the active composition of the catalyst, and TiO₂ (including the impurities mentioned herein) preferably forms about 40 to 99% by weight of the active composition of the catalyst. The inventive catalysts, in addition to TiO₂, preferably also comprise vanadium oxide. In addition, oxides of niobium and/or antimony and/or further components, for example Cs and/or P, are optionally also present. With regard to the further components of the catalytically active composition of the inventive catalysts (in addition to TiO₂) reference may in principle be made to the compositions which are described in the relevant prior art and are familiar to those skilled in the art. As stated above, they are mainly catalyst systems which, in addition to titanium oxide(s), comprise oxides of vanadium. Examples of catalysts are described, for example, in EP 0 964 744 B1, whose disclosure on this subject is hereby incorporated explicitly by reference into the description.

In a preferred inventive embodiment, the catalysts or their active composition comprise:

| | |
|---|---|
| V₂O₅ | 0-30% by weight, |
| | in particular 1-30% by weight |
| Sb₂O₃ or Sb₂O₅ | 0-10% by weight |
| Cs | 0-2% by weight |
| P | 0-5% by weight |
| Nb | 0-5% by weight |
| Further components such as | 0-5% weight |
| Ba, W, Mo, Y, Ce, Mg, Sn, Bi, | |
| Fe, Ag, Co, Ni, Cu, Au, Sn, | |
| Zr etc. | |
| TiO₂ (including the | 40 to 99% by weight, |
| impurities) | in particular remainder up to |
| | 100% by weight |

In particular, the prior art describes a series of promoters for enhancing the productivity of the catalysts, which can likewise be used in the inventive catalyst. These include the alkali metals and alkaline earth metals, thallium, antimony, phosphorus, iron, niobium, cobalt, molybdenum, silver, tungsten, tin, lead, zirconium, copper, gold and/or bismuth, and also mixtures of two or more of the aforementioned components. For example, DE 21 59 441 A describes a catalyst which, in addition to titanium dioxide of the anatase modification, consists of 1 to 30% by weight of vanadium pentoxide and zirconium dioxide. It is possible via the individual promoters to influence the activity and selectivity of the catalysts, especially by lowering or increasing the activity. The selectivity-increasing promoters include, for example, the alkali metal oxides, whereas oxidic phosphorus compounds, especially phosphorus pentoxide, can lower the activity of the catalyst at the cost of selectivity depending on the degree of promotion.

In the context of the present invention, it has been found that, surprisingly, the effect of the sulphur and/or phosphorus present in the TiO₂ used, if appropriate after the above-described washing procedure, is different to that in the case of separate addition of the sulphur and/or phosphorus during the catalyst synthesis (as additional sulphur- or phosphorus-containing component(s) of the catalyst apart from the sulphur and phosphorus fractions present in the TiO₂). The quantitative statements made herein for such additional sulphur- or phosphorus-containing components of the catalyst therefore do not include the sulphur or phosphorus contamination of the TiO₂ used. The same applies to the desired niobium content of the titanium dioxide used in accordance with the invention. It is suspected, without the invention being restricted to this assumption, that the sulphur and/or phosphorus present in accordance with the invention at an only minor impurity in the TiO₂ is strongly bonded to the TiO₂ or even incorporated into the lattice. The further sulphur- and/or phosphorus-containing components optionally added in the preparation of the inventive catalysts are apparently adsorbed only partly on the surface of the TiO₂, while a majority can interact with the catalytically active constituents such as the oxides of vanadium or any other oxides present. The same applies to niobium.

For the preparation of the catalysts described herein, the prior art describes numerous suitable processes, so that a detailed description is in principle not required here. It is possible to select any type of catalyst which is customary and familiar to those skilled in the art, including unsupported catalysts and coated catalysts which comprise an inert support and at least one layer applied thereto with a catalytically active composition comprising the TiO₂ used in accordance with the invention. For the preparation of coated catalysts, reference can be made, for example, to the process described in DE-A-16 42 938 or DE-A 17 69 998, in which a solution or suspension, comprising an aqueous and/or an organic solvent, of the components of the catalytically active composition and/or their precursor compounds (frequently referred to as “slurry”) are sprayed onto the support material in a heated coating drum at elevated temperature until the desired content of catalytically active composition, based on the total catalyst weight, has been attained.

Preference is given to preparing coated catalysts by the application of a thin layer of 50 to 500 μm of the active components to an inert support (for example U.S. Pat. No. 2,035,606). Useful supports have been found to be in particular spheres or hollow cylinders. These shaped bodies give rise to a high packing density at low pressure drop and reduce the risk of formation of packing faults when the catalyst is charged into the reaction tubes.

The molten and sintered shaped bodies have to be heat-resistant within the temperature range of the reaction as it proceeds. As stated above, examples of useful substances include silicon carbide, steatite, quartz, porcelain, SiO₂, Al₂O₃ or alumina.

The advantage of the coating of support bodies in a fluidized bed is the high uniformity of the layer thickness, which plays a crucial role for the catalytic performance of the catalyst. A particularly uniform coating is obtained by spraying a suspension or solution of the active components onto the heated support at 80 to 200° C. in a fluidized bed, for example according to DE 12 80 756, DE 198 28 583 or DE 197 09 589. In contrast to the coating in coating drums, it is also possible, when hollow cylinders are used as the support, to uniformly coat the inside of the hollow cylinders in the fluidized bed processes mentioned. Among the abovementioned fluidized bed processes, the process according to DE 197 09 589 in particular is advantageous, since the predominantly horizontal, circular motion of the supports achieves not only a uniform coating but also low abrasion of apparatus parts.

For the coating operation, the aqueous solution or suspension of the active components and of an organic binder, preferably a copolymer of vinyl acetate/vinyl laurate, vinyl acetate/ethylene or styrene/acrylate, is sprayed via one or more nozzles onto the heated, fluidized support. It is particularly favourable to introduce the spray liquid at the point of the highest product speed, as the result of which the sprayed substance can be distributed uniformly in the bed. The spray operation is continued until either the suspension has been consumed or the required amount of active components has been applied on the support.

In a particularly preferred inventive embodiment, the catalytically active composition of the inventive catalyst comprising the TiO₂ as defined herein is applied in a moving bed or fluidized bed with the aid of suitable binders, so as to obtain a coated catalyst. Suitable binders include organic binders familiar to those skilled in the art, preferably copolymers, advantageously in the form of an aqueous dispersion, of vinyl acetate/vinyl laurate, vinyl acetate/acrylate, styrene/acrylate, vinyl acetate/maleate and vinyl acetate/ethylene. Particular preference is given to using an organic polymeric or copolymeric adhesive, in particular a vinyl acetate copolymer adhesive, as the binder. The binder used is added in customary amounts to the catalytically active composition, for example at about 10 to 20% by weight based on the solids content of the catalytically active composition. For example, reference can be made to EP 744 214. when the catalytically active composition is applied at elevated temperatures of about 150° C., it is also possible, as is known from the prior art, to apply to the support without organic binders. Coating temperatures which can be used when the above-specified binders are used are, according to DE 21 06 796, for example, between about 50 and 450° C. The binders used burn off within a short time in the course of baking-out of the catalyst when the charged reactor is put into operation. The binders serve primarily to reinforce the adhesion of the catalytically active composition on the support and to reduce attrition in the course of transport and charging of the catalyst.

Further possible processes for preparing coated catalysts for the catalytic gas phase oxidation of aromatic hydrocarbons to carboxylic acids and/or carboxylic anhydrides have been described, for example, in WO 98/00778 and EP-A 714 700. According to these, from a solution and/or a suspension of the catalytically active metal oxides and/or their precursor compounds, optionally in the presence of assistants for the catalyst preparation, a powder is prepared initially and is subsequently, for the catalyst preparation on the support, optionally after conditioning and also optionally after heat treatment, applied in coating form to generate the catalytically active metal oxides, and the support coated in this way is subjected to a heat treatment to generate the catalytically active metal oxides or to a treatment to remove volatile constituents.

Suitable conditions for carrying out a process for preparing phthalic anhydride from o-xylene and/or naphthalene are equally familiar to those skilled in the art from the prior art. In particular, reference is made to the comprehensive description in K. Towae, W. Enke, R. Jäckh, N. Bhargana “Phthalic Acid and Derivatives” in Ullmann's Encyclopedia of Industrial Chemistry Vol. A. 20, 1992, 181, and this is hereby incorporated by reference. For example, the boundary conditions known from the above reference of WO-A 98/37967 or of WO 99/61433 may be selected for the steady operating state of the oxidation.

To this end, the catalysts are initially charged into the reaction tubes of the reactor, which are thermostated externally to the reaction temperature, for example by means of salt melts. The reaction gas is passed over the catalyst charge thus prepared at temperatures of generally 300 to 450° C., preferably 320 to 420° C., and more preferably of 340 to 400° C., and at an elevated pressure of generally 0.1 to 2.5 bar, preferably of 0.3 to 1.5 bar, with a space velocity of generally 750 to 5000 h⁻¹.

The reaction gas fed to the catalyst is generally generated by mixing a molecular oxygen-containing gas which, apart from oxygen, may also comprise suitable reaction moderators and/or diluents such as steam, carbon dioxide and/or nitrogen with the aromatic hydrocarbon to be oxidized, and the molecular oxygen-containing gas may generally contain 1 to 100 mol %, preferably 2 to 50 mol % and more preferably 10 to 30 mol %, of oxygen, 0 to 30 mol %, preferably 0 to 10 mol %, of steam, and 0 to 50 mol %, preferably 0 to 1 mol %, of carbon dioxide, remainder nitrogen. To generate the reaction gas, the molecular oxygen-containing gas is generally charged with 30 to 150 g per m³ (STP) of gas of the aromatic hydrocarbon to be oxidized.

In a particularly preferred inventive embodiment, the catalyst has an active composition content between about 7 and 12% by weight, preferably between 8 and 10% by weight. The active composition (catalytically active composition) preferably contains between 5 and 15% by weight of V₂O₅, 0 and 4% by weight of Sb₂O₃, 0.2 and 0.75% by weight of Cs, 0 and 3% by weight of Nb₂O₅. In addition to the aforementioned components, the remainder of the active composition consists of TiO₂ to an extent of at least 90% by weight, preferably at least 95% by weight, more preferably at least 98% by weight, in particular at least 99% by weight, more preferably at least 99.5% by weight, in particular 100% by weight. Such an inventive catalyst may, for example, advantageously be used in a two-zone or multizone catalyst as the first catalyst zone disposed toward the gas inlet side.

In a particularly preferred inventive embodiment, the BET surface area of the catalyst is between 15 and about 25 m²/g. It is further preferred that such a first catalyst zone has a length fraction of about 40 to 60% in the total length of all catalyst zones present (total length of the catalyst bed present).

In a further preferred inventive embodiment, the catalyst has an active composition content of about 6 to 11% by weight, in particular 7 to 9% by weight. The active composition contains preferably 5 to 15% by weight of V₂O₅, 0 to 4% by weight of Sb₂O₃, 0.05 to 0.3% by weight of Cs, 0 to 2% by weight of Nb₂O₅ and 0-2% by weight of phosphorus. In addition to the aforementioned components, the remainder of the active composition consists of TiO₂ to an extent of at least 90% by weight, preferably at least 95% by weight, more preferably at least 98% by weight, in particular at least 99% by weight, more preferably at least 99.5% by weight, in particular 100% by weight. Such an inventive catalyst may, for example, be used advantageously as the second catalyst zone, i.e. downstream of the first catalyst zone disposed toward the gas inlet side (see above). It is preferred that the catalyst has a BET surface area between about 15 and 25 m²/g. It is further preferred that this second zone has a length fraction of about 10 to 30% of the total length of all catalyst zones present.

In a further inventive embodiment, the catalyst has an active composition content between about 5 and 10% by weight, in particular between 6 and 8% by weight. The active composition (catalytically active composition) preferably contains 5 to 15% by weight of V₂O₅, 0 to 4% by weight of Sb₂O₃, 0 to 0.1% by weight of Cs, 0 to 1% by weight of Nb₂O₅ and 0-2% by weight of phosphorus. In addition to the aforementioned components, the remainder of the active composition consists of TiO₂ to an extent of at least 90% by weight, preferably at least 95% by weight, more preferably at least 98% by weight, in particular at least 99% by weight, more preferably at least 99.5% by weight, in particular 100% by weight. Such a catalyst may be used, for example, advantageously as the third (or last) catalyst zone disposed downstream of the above-described second catalyst zone. Preference is given to a BET surface area of the catalyst which is somewhat higher than that of the layers disposed closer to the gas inlet side, in particular in the range between about 25 and about 45 m²/g. It is further preferred that such a third catalyst zone has a length fraction of about 10 to 50% of the total length of all catalyst zones present.

It has also been found that, surprisingly, the preferred multizone or multilayer catalysts, especially having three or more layers, can be used particularly advantageously when the individual catalyst zones are present in a particular length ratio relative to one another.

In a particularly preferred inventive embodiment, the first catalyst zone disposed toward the gas inlet side has a length fraction, based on the total length of the catalyst bed, of at least 40%, in particular at least 45%, more preferably at least 50%. It is especially preferred that the fraction of the first catalyst zone in the total length of the catalyst bed is between 40 and 70%, in particular between 40 and 55%, more preferably between 40 and 52%.

In a particularly preferred 4-zone catalyst, the first catalyst zone has a length fraction, based on the total length of the catalyst bed, between about 10% and 20%. The length fraction of the second catalyst zone is preferably between about 40% and 60%, based on the total length of the catalyst bed. The length fraction of the third and fourth catalyst zones is preferably in each case between about 15% and 40%, based on the total length of the catalyst bed.

The second zone takes up preferably about 10 to 40%, in particular about 10 to 30%, of the total length of the catalyst bed. It has also been found that, surprisingly, a ratio of the length of the third catalyst zone to the length of the second catalyst zone between about 1 and 2, in particular between about 1.2 and 1.7, more preferably between 1.3 and 1.6, affords particularly good results with regard to the economic viability, such as the efficiency of raw material utilization and productivity of the catalyst.

It has been found that the above selection of length fractions of the individual catalyst zones enables particularly favourable positioning of the hotspot, especially within the first zone, and good temperature control for preventing excessively high hotspot temperatures even in the case of prolonged operating time of the catalyst. This improves the yield, especially based on the lifetime of the catalyst.

Temperature management in the gas phase oxidation of o-xylene to phthalic anhydride is sufficiently well known to those skilled in the art from the prior art, and reference may be made, for example, to DE 100 40 827 A1.

Moreover, it is preferred in accordance with the invention that, when the catalyst prepared in accordance with the invention is used in a multizone catalyst bed for preparing phthalic anhydride, the content of alkali metals in the catalyst zones falls from the gas inlet side to the gas outlet side. In a particularly preferred embodiment, the alkali metal content, preferably the Cs content (calculated as Cs), in the second catalyst zone is lower than in the first catalyst zone, and in the third catalyst zone is lower than in the second catalyst zone (and preferably, if appropriate, zones which follow the third zone). More preferably, the Cs content (calculated as Cs) in the catalyst therefore increases from zone to zone in gas flow direction. In a preferred embodiment, the third (and preferably also any downstream catalyst zones) does not comprise any Cs. Preferably:

Cs content_{1st zone}>Cs content_{2nd zone}> . . . >Cs content_{last zone}.

More preferably, the last catalyst zone does not comprise any Cs.

In a particularly preferred embodiment, only the last catalyst zone comprises phosphorus. In a further particularly preferred embodiment, no phosphorus is present in the active composition in the 1st zone and in the 2nd zone, and in a 4-zone catalyst preferably not in the 3rd catalyst zone either. (“No phosphorus is present” means that no phosphorus was added actively to the active composition in the course of preparation.)

It has also been found that, surprisingly, particularly favourable three- or multizone catalysts can be obtained in many cases when the active composition content decreases from the first catalyst zone disposed toward the gas inlet side to the catalyst zone disposed toward the gas outlet side. If has been found to be advantageous that the first catalyst zone has an active composition content between about 7 and 12% by weight, in particular between about 8 and 11% by weight, the second catalyst zone an active composition content between about 6 and 11% by weight, in particular between about 7 and 10% by weight, and the third catalyst zone an active composition content between about 5 and 10% by weight, in particular between about 6 and 9% by weight.

The expressions “first, second and third catalyst zone” are used in connection with the present invention as follows: the first catalyst zone refers to the catalyst zone disposed toward the gas inlet side. In the inventive catalyst, another two catalyst zones are present toward the gas outlet side, and are referred to as the second and third catalyst zone respectively. The third catalyst zone is closer to the gas outlet side than the second catalyst zone.

In a particularly preferred inventive embodiment, the catalyst has three or four catalyst zones. In a 3-zone catalyst, the third catalyst zone is at the gas outlet side. The presence of additional catalyst zones downstream in gas flow direction of the first catalyst zone is, however, not ruled out. For example, in a further particularly preferred inventive embodiment, the third catalyst zone as defined herein may also be followed by a fourth catalyst zone (preferably having an equal or even lower active composition content than the third catalyst zone).

According to the invention, in one embodiment, the active composition content can decrease between the first and the second catalyst zone and/or between the second and the third catalyst zone. In a particularly preferred inventive embodiment, the active composition content decreases between the second and the third catalyst zone. In a further preferred inventive embodiment, the BET surface area increases from the first catalyst zone disposed toward the gas inlet side to the third catalyst zone disposed toward the gas outlet side. Preferred ranges for the BET surface area are 15 to 25 m²/g for the first catalyst zone, 15 to 25 m²/g for the second catalyst zone and 25 to 45 m²/g for the third catalyst zone.

In many cases, it is preferred in accordance with the invention that the BET surface area of the first catalyst zone is lower than the BET surface area of the third catalyst zone. Particularly advantageous catalysts are also obtained when the BET surface areas of the first and of the second catalyst zones are equal, while the BET surface area of the third catalyst zone is larger in comparison. The catalyst activity toward the gas inlet side is, in a preferred inventive embodiment, lower than the catalyst activity toward the gas outlet side.

It is also preferred that at least 0.05% by weight of the catalytically active composition is formed by at least one alkali metal, calculated as alkali metal(s). Particular preference is given to using caesium as the alkali metal.

In addition, according to the inventor's results, in one embodiment, it is preferred that the catalyst comprises niobium in a total amount of 0.01 to 2% by weight, in particular 0.5 to 1% by weight, of the catalytically active composition.

The inventive catalysts are typically thermally treated or calcined (conditioned) before use. It has been found to be advantageous when the catalyst is calcined at at least 390° C. for at least 24 hours, in particular at at 400° C. for between 24 and 72 hours, in an O₂-containing gas, especially in air. The temperatures should preferably not exceed about 500° C., in particular about 470° C. In principle, however, other calcination conditions which appear to be suitable to those skilled in the art are not ruled out.

In a further aspect, the present invention relates to a process for preparing a catalyst according to one of the preceding claims, comprising the following steps:
a. providing a catalytically active composition as defined herein, comprising the TiO₂ characterized in detail above;
b. providing an inert support, especially a shaped inert body,
c. applying the catalytically active composition to the inert support, especially in a fluidized bed or a moving bed.

It is then preferably dried and calcined. In a further aspect, the present invention also relates to the use of titanium dioxide as defined above for preparing a catalyst, especially for gas phase oxidation of hydrocarbons, preferably for gas phase oxidation of o-xylene and/or naphthalene to phthalic anhydride.

In a further aspect, the present invention relates to a process for gas phase oxidation of at least one hydrocarbon, in which:

a) a catalyst comprising titanium dioxide as described herein is provided;

b) the catalyst is contacted with a gas stream which comprises the at least one hydrocarbon and oxygen,

in order to bring about the gas phase oxidation of the at least one hydrocarbon. In a particularly preferred aspect, the process is a process for preparing phthalic anhydride from o-xylene and/or naphthalene.

### Methods

To determine the parameters of the catalysts, the methods below are used:

1. BET Surface Area:

The determination is effected by the BET method according to DIN 66131; a publication of the BET method can also be found in J. Am. Chem. Soc. 60, 309 (1938).

2. Pore Radius Distribution:

The pore radius distribution and the pore volume of the TiO₂ used were determined by means of mercury porosimetry to DIN 66133; maximum pressure: 2000 bar, Porosimeter 4000 (from Porotec, Germany), according to the manufacturer's instructions.

3. Primary Crystal Sizes:

The primary crystal sizes were determined by powder X-ray diffractometry. The analysis was carried out with an instrument from Bruker, Germany: BRUKER AXS-D4 Endeavor. The resulting X-ray diffractograms were recorded with the “DiffracPlus D4 Measurement” software package according to the manufacturer's instructions, and the half-height width of the 100% refraction was evaluated with the “DiffracPlus Evaluation” software by the Debye-Scherrer formula according to the manufacturer's instructions in order to determine the primary crystal size.

4. Particle Sizes:

The particle sizes were determined by the laser diffraction method with a Fritsch Particle Sizer Analysette 22 Economy (from Fritsch, Germany) according to the manufacturer's instructions, also with regard to the sample pretreatment: the sample is homogenized in deionized water without addition of assistants and treated with ultrasound for 5 minutes.

5. Determination of the Impurities of the TiO₂:

The chemical impurities of the TiO₂, especially the contents of S, P, Nb, were determined to DIN ISO 9964-3. Thus, the contents can be determined by means of ICP-AES (Inductively Coupled Plasma Atomic Emission Spectroscopy) and, if appropriate in the case of alkali metals, added up to give the total alkali metal content of the TiO₂.

6. Bulk Density:

The bulk density was determined with the aid of the TiO₂ used to prepare the catalyst (dried at 150° C. under reduced pressure, uncalcined). The resulting values from three determinations were averaged.

The bulk density was determined by introducing 100 g of the TiO₂ material into a 1000 ml container and shaken for approx. 30 seconds.

A measuring cylinder (capacity exactly 100 ml) is weighed empty to 10 mg. Above it, the powder funnel is secured over the opening of the cylinder using a clamp stand and clamp. After the stopwatch has been started, the measuring cylinder is charged with the TiO₂ material within 15 seconds. The spatula is used to constantly supply more filling material, so that the measuring cylinder is always slightly overfilled. After 2 minutes, the spatula is used to level off the excess, care being taken that no pressing forces compress the material in the cylinder. The filled measuring cylinder is brushed off and weighed.

The bulk density is reported in g/l.

The BET surface area, the pore radius distribution and the pore volume, and also the primary crystal sizes and the particle size distribution were determined for the titanium dioxide in each case on the uncalcined material dried at 150° C. under reduced pressure.

The data in the present description with regard to the BET surface areas of the catalysts or catalyst zones also relate to the BET surface areas of the TiO₂ material used in each case (dried at 150° C. under reduced pressure, uncalcined, see above).

In general, the BET surface area of the catalyst is determined by virtue of the BET surface area of the TiO₂ used, although the addition of further catalytically active components does change the BET surface area to a certain extent. This is familiar to those skilled in the art.

The active composition content (content of the catalytically active composition, without binder) relates in each case to the content (in % by weight) of the catalytically active composition in the total weight of the catalyst including support in the particular catalyst zone, measured after conditioning at 400° C. over 4 h.

The invention will now be illustrated in detail with reference to the non-restrictive examples which follow:

### EXAMPLES

### Example 1

### Preparation of Catalyst A (Comparative)

To prepare the catalyst A having an active composition content of 8% by weight and the composition of 7.5% by weight of vanadium pentoxide, 3.2% by weight of antimony trioxide, 0.40% by weight of caesium (calculated as caesium), 0.2% by weight of phosphorus (calculated as phosphorus) and remainder titanium dioxide, 2200 g of steatite bodies in the form of hollow cylinders of size 8×6×5 mm were coated in a so-called fluidized bed coater with a suspension composed of 15.1 g of vanadium pentoxide, 6.4 g of antimony trioxide, 1.1 g of caesium sulphate, 1.5 g of ammonium dihydrogenphosphate, 178.6 g of titanium dioxide having a BET surface area of 19 m²/g (from Nano Co. Ltd., 1108-1 Bongkok Sabong, Jinju, Kyoungnam 660-882 Korea, trade name NT22) and the following chemical impurities:

| | |
|---|---|
| S: | 1450 ppm |
| P: | 760 ppm |
| Nb: | 1180 ppm |
| sum(alkali metals): | 280 ppm |

120.5 g of binder composed of a 50% dispersion of water and vinyl acetate/ethylene copolymer (Vinnapas® EP 65 W, from Wacker) and 1000 g of water at a temperature of 70° C. The active composition was applied in the form of thin layers.

### Example 2

### Preparation of Catalyst B (Inventive)

Before the actual preparation of catalyst B, 200 g of the TiO₂ according to Example 1 were washed, in several washing and filtering steps in each case, first with 1 molar nitric acid, bidistilled water, 1 molar aqueous ammonia and finally again with bidistilled water, in each case with stirring for 12 h, and filtered off. Subsequently, the sample was dried. The washed TiO₂ material had the following chemical impurities:

| | |
|---|---|
| S: | 850 ppm |
| P: | 450 ppm |
| Nb: | 1170 ppm |
| sum(alkali metals): | 250 ppm |

To prepare catalyst B having an active composition content of 8% by weight and the composition of 7.5% by weight of vanadium pentoxide, 3.2% by weight of antimony trioxide, 0.40% by weight of caesium (calculated as caesium), 0.2% by weight of phosphorus (calculated as phosphorus) and remainder titanium dioxide, 2200 g of steatite bodies in the form of hollow cylinders of size 8×6×5 mm were coated in a so-called fluidized bed coater with a suspension of 15.1 g of vanadium pentoxide, 6.4 g of antimony trioxide, 1.1 g of caesium sulphate, 1.5 g of ammonium dihydrogenphosphate, 178.6 g of the titanium dioxide washed as described above (BET surface area 19 m²/g), 120.5 g of binder composed of a 50% dispersion of water and vinyl acetate/ethylene copolymer (Vinnapas® EP 65 W, from Wacker) and 1000 g of water at a temperature of 70° C. The active composition was applied in the form of thin layers.

### Example 3

### Preparation of Catalyst C (Inventive)

Before the actual preparation of catalyst C, 200 g of the TiO₂ already washed according to Example 2 were washed, in each case in several washing and filtering steps, first with 1 molar nitric acid, bidistilled water, 1 molar aqueous ammonia and finally again with bidistilled water, in each case for 12 h with stirring, and filtered off. Subsequently, the sample was dried. The washed TiO₂ material had the following chemical impurities:

| | |
|---|---|
| S: | 290 ppm |
| P: | 260 ppm |
| Nb: | 1150 ppm |
| sum(alkali metals): | 230 ppm |

To prepare catalyst C with an active composition content of 8% by weight and the composition of 7.5% by weight of vanadium pentoxide, 3.2% by weight of antimony trioxide, 0.40% by weight of caesium (calculated as caesium), 0.2% by weight of phosphorus (calculated as phosphorus) and remainder titanium dioxide, 2200 g of steatite bodies in the form of hollow cylinders of size 8×6×5 mm were then coated in a so-called fluidized bed coater with a suspension of 15.1 g of vanadium pentoxide, 6.4 g of antimony trioxide, 1.1 g of caesium sulphate, 1.5 g of ammonium dihydrogenphosphate, 178.6 g of the titanium dioxide washed as described above (BET surface area 19 m²/g), 120.5 g of binder composed of a 50% dispersion of water and vinyl acetate/ethylene copolymer (Vinnapas® EP 65 W, from Wacker) and 1000 g of water at a temperature of 70° C. The active composition was applied in the form of thin layers.

### Example 4

### Preparation of Catalyst D (Inventive)

Before the actual preparation of catalyst D, 200 g of TiO₂ already washed according to Example 3 were washed, in each case in several washing and filtering steps, first with 1 molar nitric acid, bidistilled water, 1 molar aqueous ammonia and finally again with bidistilled water, in each case with stirring for 12 h, and filtered off. Finally, the sample was dried. The washed TiO₂ material had the following chemical impurities:

| | |
|---|---|
| S: | 140 ppm |
| P: | 200 ppm |
| Nb: | 1160 ppm |
| sum (alkali metals) | 230 ppm |

To prepare catalyst D with an active composition content of 8% by weight and the composition of 7.5% by weight of vanadium pentoxide, 3.2% by weight of antimony trioxide, 0.40% by weight of caesium (calculated as caesium), 0.2% by weight of phosphorus (calculated as phosphorus) and remainder titanium dioxide, 2200 g of steatite bodies in the form of hollow cylinders of size 8×6×5 mm were then coated in a so-called fluidized bed coater with a suspension of 15.1 g of vanadium pentoxide, 6.4 g of antimony trioxide, 1.1 g of caesium sulphate, 1.5 g of ammonium dihydrogenphosphate, 178.6 g of the titanium dioxide washed as described above (BET surface area 19 m²/g), 120.5 g of binder composed of a 50% dispersion of water and vinyl acetate/ethylene copolymer (Vinnapas® EP 65 W, from Wacker) and 1000 g of water at a temperature of 70° C. The active composition was applied in the form of thin layers.

### Example 5

### Determining the Catalytic Performance Data of Catalysts A, B, C and D

A 120 cm-long reaction tube with an internal diameter of 24.8 mm is filled to a length of 80 cm with 40 g of catalyst A diluted with 200 g of steatite rings of dimensions 8×6×5 mm to prevent hotspots. The reaction tube is disposed in a liquid salt melt which can be heated to temperatures up to 450° C. Within the catalyst bed is disposed a 3 mm protective tube with installed thermoelement, by means of which the catalyst temperature can be indicated over the complete catalyst combination. To determine the catalytic performance data, 60 g/m³ (STP) of o-xylene (purity 99.9%) with a maximum of 400 l (STP) of air/h are passed through catalyst A. Subsequently, the salt bath temperature is adjusted to the effect that the o-xylene conversion is between 55 and 65%. The results of the test run are listed in Table 1.

The procedure is repeated in parallel test runs with catalysts B, C and D. The results of the test runs are listed in Table 1.

TABLE 1

| | | Salt bath | C₈ | PA | COₓ | MA |
|---|---|---|---|---|---|---|
| | Conversion | temperature | selectivity | selectivity | selectivity | selectivity |
| Example | [%] | [° C.] | [mol %] | [mol %] | [mol %] | [mol %] |
| Cat. A (Ex. 1) | 59.4 | 380 | 82.8 | 70.9 | 12.5 | 3.0 |
| Cat. B (Ex. 2) | 63.2 | 380 | 84.5 | 74.7 | 11.3 | 2.1 |
| Cat. C (Ex. 3) | 62.8 | 380 | 86.0 | 76.1 | 10.5 | 1.4 |
| Cat. D (Ex. 4) | 64.4 | 380 | 86.6 | 76.6 | 10.2 | 1.5 |

C₈ selectivity: selectivity with regard to all products of value having 8 carbon atoms (phthalic anhydride, phthalide, o-tolylaldehyde, o-toluic acid)
COₓ: sum of carbon monoxide and dioxide in the offgas stream
PA: phthalic anhydride;
MA: maleic anhydride;
Cat.: catalyst

It is clearly evident from Table 1 that both the conversion and the C₈ and the PA selectivity for the inventive catalysts (catalysts B, C and D) are significantly higher than for the comparative material (catalyst A). Moreover, the formation of MA as a by-product for the inventive catalysts is significantly lower than for the comparative material. It is also found that the improved properties of the catalysts are not attributable to a different alkali metal content of the materials used, since catalysts A to D do not differ greatly in this regard.

### Example 6

### Preparation of Catalyst E (Comparative)

To prepare the catalyst E having an active composition content of 8% by weight and the composition of 7.5% by weight of vanadium pentoxide, 3.2% by weight of antimony trioxide, 0.40% by weight of caesium (calculated as caesium), 0.2% by weight of phosphorus (calculated as phosphorus) and remainder titanium dioxide, 2200 g of steatite bodies in the form of hollow cylinders of size 8×6×5 mm were coated in a so-called fluidized bed coater with a suspension composed of 15.1 g of vanadium pentoxide, 6.4 g of antimony trioxide, 1.1 g of caesium sulphate, 1.5 g of ammonium dihydrogenphosphate, 178.6 g of a commercially available titanium dioxide having a BET surface area of 20 m²/g and the following chemical impurities:

| | |
|---|---|
| S: | 2230 ppm |
| P: | 880 ppm |
| Nb: | 1530 ppm |

120.5 g of binder (see Example 1) and 1000 g of water at a temperature of 70° C. The active composition was applied in the form of thin layers.

### Example 7

### Preparation of Catalyst F (Inventive)

To prepare the catalyst F having an active composition content of 8% by weight and the composition of 7.5% by weight of vanadium pentoxide, 3.2% by weight of antimony trioxide, 0.40% by weight of caesium (calculated as caesium), 0.2% by weight of phosphorus (calculated as phosphorus) and remainder titanium dioxide, 2200 g of steatite bodies in the form of hollow cylinders of size 8×6×5 mm were coated in a so-called fluidized bed coater with a suspension composed of 15.1 g of vanadium pentoxide, 6.4 g of antimony trioxide, 1.1 g of caesium sulphate, 1.5 g of ammonium dihydrogenphosphate, 178.6 g of titanium dioxide having a BET surface area of 19 m²/g (obtained by washing steps according to Example 2 from another commercially available TiO₂) and the following chemical impurities:

| | |
|---|---|
| S: | 120 ppm |
| P: | 220 ppm |
| Nb: | 1160 ppm |

120.5 g of binder (see Example 1) and 1000 g of water at a temperature of 70° C. The active composition was applied in the form of thin layers.

### Example 8

### Preparation of Catalyst G (Inventive)

To prepare the catalyst G having an active composition content of 8% by weight and the composition of 7.5% by weight of vanadium pentoxide, 3.2% by weight of antimony trioxide, 0.40% by weight of caesium (calculated as caesium), 0.2% by weight of phosphorus (calculated as phosphorus) and remainder titanium dioxide, 2200 g of steatite bodies in the form of hollow cylinders of size 8×6×5 mm were coated in a so-called fluidized bed coater with a suspension composed of 15.1 g of vanadium pentoxide, 6.4 g of antimony trioxide, 1.1 g of caesium sulphate, 1.5 g of ammonium dihydrogenphosphate, 178.6 g of titanium dioxide having a BET surface area of 20 m²/g (obtained by washing steps according to Example 2 from another commercially available TiO₂) and the following chemical impurities:

| | |
|---|---|
| S: | 250 ppm |
| P: | 240 ppm |
| Nb: | 1350 ppm |

120.5 g of binder (see Example 1) and 1000 g of water at a temperature of 70° C. The active composition was applied in the form of thin layers.

### Example 9

### Preparation of Catalyst H (Inventive)

To prepare the catalyst H having an active composition content of 8% by weight and the composition of 7.5% by weight of vanadium pentoxide, 3.2% by weight of antimony trioxide, 0.40% by weight of caesium (calculated as caesium), 0.2% by weight of phosphorus (calculated as phosphorus) and remainder titanium dioxide, 2200 g of steatite bodies in the form of hollow cylinders of size 8×6×5 mm were coated in a so-called fluidized bed coater with a suspension composed of 15.1 g of vanadium pentoxide, 6.4 g of antimony trioxide, 1.1 g of caesium sulphate, 1.5 g of ammonium dihydrogenphosphate, 178.6 g of titanium dioxide having a BET surface area of 19 m²/g (obtained by washing steps according to Example 2 from another commercially available TiO₂) and the following chemical impurities:

| | |
|---|---|
| S: | 480 ppm |
| P: | 620 ppm |
| Nb: | 1800 ppm |

120.5 g of binder (see Example 1) and 1000 g of water at a temperature of 70° C. The active composition was applied in the form of thin layers.

### Example 10

### Determination of the Catalytic Performance Data of Catalysts E to H

A 120 cm-long reaction tube with an internal diameter of 24.8 mm is filled to a length of 80 cm with 40 g of catalyst E diluted with 200 g of steatite rings of dimensions 8×6×5 mm to prevent hotspots. The reaction tube is disposed in a liquid salt melt which can be heated to temperatures up to 450° C. Within the catalyst bed is disposed a 3 mm protective tube with installed thermoelement, by means of which the catalyst temperature can be indicated over the complete catalyst combination. To determine the catalytic performance data, 60 g/m³ (STP) of o-xylene (purity 99.9%) with a maximum of 400 l (STP) of air/h are passed through catalyst A. Subsequently, the salt bath temperature is adjusted to the effect that the o-xylene conversion is between 55 and 65%. The results of the test run are listed in Table 2.

The procedure is repeated in parallel test runs with catalysts F, G and H. The results of the test runs are listed in Table 2.

TABLE 2

| | | Salt bath | C₈ | PA | COₓ | MA |
|---|---|---|---|---|---|---|
| | Conversion | temperature | selectivity | selectivity | selectivity | selectivity |
| Example | [%] | [° C.] | [mol %] | [mol %] | [mol %] | [mol %] |
| Cat. E (Ex. 6) | 57.4 | 376 | 83.2 | 72.1 | 12.5 | 3.3 |
| Cat. F (Ex. 7) | 64.8 | 376 | 87.1 | 78.3 | 10.3 | 1.8 |
| Cat. G (Ex. 8) | 63.4 | 376 | 86.9 | 77.2 | 10.5 | 2.2 |
| Cat. H (Ex. 9) | 60.6 | 376 | 85.2 | 75.6 | 11.2 | 2.8 |

C₈ selectivity: selectivity with regard to all products of value having 8 carbon atoms (phthalic anhydride, phthalide, o-tolylaldehyde, o-toluic acid)
COₓ: sum of carbon monoxide and dioxide in the offgas stream
PA: phthalic anhydride
MA: maleic anhydride

### Example 11

### Preparation of an Inventive Three-Layer Catalyst

An inventive three-layer catalyst can be obtained, for example, as follows:

To prepare catalyst J having an active composition content of 9% by weight and the composition of 7.5% by weight of vanadium pentoxide, 3.2% by weight of antimony trioxide, 0.40% by weight of caesium (calculated as caesium), 0.2% by weight of phosphorus (calculated as phosphorus) and remainder titanium dioxide (as in Example 3), 2200 g of steatite bodies in the form of hollow cylinders of size 8×6×5 mm were coated in a so-called fluidized bed coater with a suspension of 17.2 g of vanadium pentoxide, 7.3 g of antimony trioxide, 1.25 g of caesium sulphate, 1.72 g of ammonium dihydrogenphosphate, 203.2 g of titanium dioxide having a BET surface area of 19 m²/g, 120 g of binder composed of a 50% dispersion of water and vinyl acetate/ethylene copolymer (Vinnapas® EP 65 W, from Wacker) and 1000 g of water at a temperature of 70° C. The active composition was applied in the form of thin layers.

To prepare catalyst K having an active composition content of 8% by weight and the composition of 7.5% by weight of vanadium pentoxide, 3.2% by weight of antimony trioxide, 0.20% by weight of caesium (calculated as caesium), 0.2% by weight of phosphorus (calculated as phosphorus) and remainder titanium dioxide (as in Example 3), 2200 g of steatite bodies in the form of hollow cylinders of size 8×6×5 mm were coated in a so-called fluidized bed coater with a suspension of 15.1 g of vanadium pentoxide, 6.4 g of antimony trioxide, 0.5 g of caesium sulphate, 1.5 g of ammonium dihydrogenphosphate, 179 g of titanium dioxide having a BET surface area of 19 m²/g, 120 g of binder composed of a 50% dispersion of water and vinyl acetate/ethylene copolymer (Vinnapas® EP 65 W, from Wacker) and 1000 g of water at a temperature of 70° C. The active composition was applied in the form of thin layers.

To prepare catalyst L having an active composition content of 8% by weight and the composition of 11% by weight of vanadium pentoxide, 0.35% by weight of phosphorus (calculated as phosphorus) and remainder titanium dioxide (as in Example 3), 2200 g of steatite bodies in the form of hollow cylinders of size 8×6×5 mm were coated in a so-called fluidized bed coater with a suspension of 22.2 g of vanadium pentoxide, 2.6 g of ammonium dihydrogenphosphate, 178.5 g of titanium dioxide having a BET surface area of 19 m²/g, 120 g of binder composed of a 50% dispersion of water and vinyl acetate/ethylene copolymer (Vinnapas® EP 65 W, from Wacker) and 1000 g of water at a temperature of 70° C. The active composition was applied in the form of thin layers.

The sequence of the catalyst zones: 140 cm of catalyst J, 60 cm of catalyst K, 90 cm of catalyst L.

### Example 12

### Catalytic Performance Data of the Inventive Three-Layer Catalyst

A 450 cm-long reaction tube is filled successively with 90 cm of catalyst L, 60 cm of catalyst K and 140 cm of catalyst J. The reaction tube is disposed in a liquid salt melt which can be heated to temperatures up to 450° C. Disposed in the catalyst bed is a 3 mm protective tube with installed thermoelement, by means of which the catalyst temperature over the complete catalyst combination can be indicated. To determine the catalytic performance data, 0 to a maximum of 70 g/m³ (STP) of o-xylene (purity 99.9%) with 3.6 m³ (STP) of air/h are passed through this catalyst combination in the sequence J, K, L, and the reaction gas, after passing through the reaction tube outlet, is passed through a condenser, in which all organic constituents of the reaction gas apart from the carbon monoxide and carbon dioxide precipitate out. The precipitated crude product is melted by means of superheated steam, collected and then weighed.

The crude yield is determined as follows.

Max. crude PA yield [% by weight]=Weighed amount of crude PA (g)×100/o-xylene feed [g]×o-xylene purity [%/100]

The results of the test run are listed in Table 3.

TABLE 3

| | | | PA quality | |
|---|---|---|---|---|
| | | | (phthalide | Hotspot |
| | Maximum | Crude | value in the | temperature |
| Example | loading | PA yield | reaction gas) | and zone |
| Example 12: | 65 g/m³ | 114.4% | <500 ppm | 438° C. |
| Catalyst | (STP) | by weight | | 65 cm |
| combination | | | | (1st zone) |
| J (140 cm) | | | | |
| K (60 cm) | | | | |
| L (90 cm) | | | | |

As is evident from Table 3, the inventive catalyst according to Example 12 exhibits a very good PA yield and PA quality. The hotspot is advantageously positioned in the first catalyst zone.

1. A catalyst for gas phase oxidation of hydrocarbons, particularly o-xylene, naphthalene or mixtures thereof, for preparing phthalic anhydride comprising a catalytically active composition comprising titanium dioxide (TiO₂) having a content of sulphur, calculated as elemental sulphur, of less than about 1000 ppm, and a BET surface area of at least 5 m²/g.

2. (canceled)

3. The catalyst of claim 1 further comprising niobium in the TiO₂, calculated as Nb in an amount greater than about 500 ppm.

4. The catalyst of claim 1, wherein the content of phosphorus in the TiO₂, calculated as elemental phosphorus, is less than 300 ppm.

5. The catalyst of claim 1, wherein the content of sulphur in the TiO₂, calculated as elemental sulphur, is less than about 750 ppm.

6. (canceled)

7. (canceled)

8. The catalyst of claim 1 further comprising an inert support and at least one layer which has been applied thereto which comprises the catalytically active composition.

9. The catalyst of claim 1, wherein the BET surface area of the TiO₂ is between about 15 and 60 m²/g.

10. The catalyst of claim 1, wherein at least some of the TiO₂ used has the following properties: (a) a BET surface area of more than 15 m²/g, (b) at least 25% of the total pore volume is formed by pores having a radius between 60 and 400 nm and (c) a primary crystal size of more than 210 ångström.

11. The catalyst of claim 1, wherein its bulk density is less than 1.0 g/ml.

12. (canceled)

13. The catalyst of claim 1, wherein the D₉₀ value of the TiO₂ is between about 0.5 and 20 μm.

14. The catalyst of claim 1, wherein 4% by weight or more of the catalytically active material comprises vanadium, calculated as vanadium pentoxide.

15. The catalyst of claim 1, wherein at least 0.05% by weight of the catalytically active material comprises at least one alkali metal, calculated as the alkali metal.

16. The catalyst of claim 1 further comprising an adhesive used for the catalytically active material comprising an organic polymer or copolymer.

17. (canceled)

18. (canceled)

19. (canceled)

20. A multizone or multilayer catalyst system comprising a first catalyst zone disposed toward a gas inlet side, a second catalyst zone disposed closer to a gas outlet side and a third catalyst zone disposed even closer to or at the gas outlet side, and wherein the catalysts present in each catalyst zones are of different composition and each have an active composition comprising TiO₂, and wherein the active composition content of the catalysts decreases from the first to the third catalyst zone, with the proviso that
a) the catalysts of the first catalyst zone have an active composition content between about 7 and 12% by weight,
b) the catalysts of the second catalyst zone have an active composition content in the range between 6 and 11% by weight and the active composition content of the catalysts of the second catalyst zone is less than or equal to the active composition content of the catalysts of the first catalyst zone, and
c) the catalysts of the third catalyst zone have an active composition content in the range between 5 and 10% by weight and the active composition content of the catalysts of the third catalyst zone is less than or equal to the active composition content of the catalysts of the second catalyst zone.

21. A multizone or multilayer catalyst system comprising a first catalyst zone disposed toward a gas inlet side, a second catalyst zone disposed closer to a gas outlet side, a third catalyst zone disposed even closer to the gas outlet side and a fourth catalyst zone disposed even closer to or at the gas outlet side, and wherein the catalysts of the catalyst zones are of different composition and each have an active composition comprising TiO₂, and wherein the active composition content of the catalysts decreases from the first to the fourth catalyst zone, with the proviso that
a) the catalysts of the first catalyst zone have an active composition content between about 7 and 12% by weight,
b) the catalysts of the second catalyst zone have an active composition content in the range between 6 and 11% by weight and the active composition content of the catalysts of the second catalyst zone is less than or equal to the active composition content of the catalysts of the first catalyst zone,
c) the catalysts of the third catalyst zone have an active composition content in the range between 5 and 10% by weight and the active composition content of the catalysts of the third catalyst zone is less than or equal to the active composition content of the catalysts of the second catalyst zone; and
d) the catalysts of the fourth catalyst zone have an active composition content in the range between 4 and 9% by weight and the active composition content of the fourth catalyst zone is less than or equal to the active composition content of the catalysts of the third catalyst zone.

22. The system of claim 20, wherein the catalyst activity of the catalyst zone or zones toward the gas inlet side is lower than the catalyst activity of the catalyst zone or zones toward the gas outlet side.

23. The system of claim 20, wherein the BET surface area of the catalysts of the first catalyst zone is lower than the BET surface area of the catalysts of the last catalyst zone.

24. The system of claim 20, wherein the proportion of the total length of the first catalyst zone to the total length of the catalyst system is between 10 and 20%.

25. The system of claim 20, wherein the proportion of the total length of the second catalyst zone to the total length of the catalyst system is between about 40 and 60%.

26. A process for preparing a catalyst for gas phase oxidation of hydrocarbons, especially for preparing phthalic anhydride by gas phase oxidation of o-xylene, naphthalene or mixtures thereof, comprising the following steps:
a. providing the catalytically active composition comprising TiO₂ of claim 1,
b. providing an inert support, especially a shaped inert body,
c. applying the catalytically active composition to the inert support, especially in a fluidized bed or a moving bed.

27. A process for gas phase oxidation of at least one hydrocarbon, comprising:
a) providing a catalyst comprising titanium dioxide and further comprising sulphur, calculated as elemental sulphur, in an amount less than about 1000 ppm, wherein the BET surface area of the catalyst is at least 5 m²/g;
b) contacting the catalyst with a gas stream which comprises the at least one hydrocarbon and oxygen,
in order to bring about the gas phase oxidation of the at least one hydrocarbon.

28. The process according to claim 27, wherein the process is for preparing phthalic anhydride from o-xylene and/or naphthalene.

29. The system of claim 20, wherein the catalysts of the third or last catalyst zone comprises from 0.05 to 0.5% by weight phosphorus.

30. The process of claim 26, wherein the catalyst is calcined or conditioned at >390° C. for at least 24 hours, in an O₂-containing gas, especially in air after application of the catalytically active compositions.

31. The process of claim 26, wherein only one TiO₂ source is used to prepare the catalyst.

32. The process of claim 27, wherein the process of gas phase oxidation is selected from the group consisting of methanol oxidation to formaldehyde, oxidative dehydrogenation of alkanes, and partial oxidation of aldehydes or alcohols to the corresponding carboxylic acids.

33. The process of claim 27, wherein the process of gas phase oxidation is selected from the group consisting of gas phase oxidation of aromatic hydrocarbons such as benzene, xylenes, naphthalene, toluene or durene to prepare carboxylic acids and/or carboxylic anhydrides; ammoxidation of alkanes and alkenes, ammoxidation of alkylaromatics and alkylheteroaromatics to the corresponding cyano compounds, especially the ammoxidation of 3-methylpyridine (β-picoline) to 3-cyanopyridine, oxidation of 3-methylpyridine to nicotinic acid, oxidation of acenaphthene to naphthalic anhydride, or of durene to pyromellitic anhydride; preparation of naphthalic anhydride from acenaphthene and preparation of cyanopyridine from alkylpyridine (picoline) by ammoxidation, for example the conversion of 3-methylpyridine to 3-cyanopyridine.
